(19) United States
(12) Patent Application Publication
(10) Pub. No.: US 2005/0249683 A1
(43) Pub. Date: Nov. 10, 2005

(54) Nail varnish composition comprising a polymer comprising units with a lower critical solution temperature

(76) Inventor: Florence L'Alloret, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/115,186

(22) Filed: Apr. 27, 2005

Related U.S. Application Data
(60) Provisional application No. 60/566,432, filed on Apr. 30, 2004.

(30) Foreign Application Priority Data
Apr. 27, 2004 (FR) 04 50800

(51) Int. Cl. A61K 7/04

(52) U.S. Cl. 424/61

(57) ABSTRACT

The present disclosure relates to nail varnish compositions comprising, in a cosmetically acceptable aqueous medium, at least one polymer chosen from water-soluble and water-dispersible polymers, wherein the at least one polymer comprises water-soluble or water-dispersible units and units with a Lower Critical Solution Temperature (LCST). The present disclosure also relates to a process for making up or caring for the nails with the compositions disclosed herein.

This application claims benefit of U.S. Provisional Application No. 60/566,432, filed Apr. 30, 2004, and also claims benefit of priority under 35 U.S.C. § 119 to French Patent Application No. 04 50800, filed Apr. 27, 2004, the contents of both of which are herein incorporated by reference.

The present disclosure relates to a nail varnish composition comprising at least one polymer chosen from water-soluble and water-dispersible polymers and comprising water-soluble or water-dispersible units, and units with a Lower Critical Solution Temperature (“LCST”). The present disclosure also relates to a makeup and/or care process for the nails.

The composition as disclosed herein can be used as a nail makeup product such as a colored nail varnish, as a base for the nails or “base coat,” as a finishing composition, also known as a “top coat,” to be applied under or over the nail makeup product, and also as a cosmetic nailcare product such as a treating base for protecting, strengthening and/or repairing the nails.

The composition as disclosed herein can be applied to human nails or to false nails.

Nail varnish compositions generally comprise solid particles such as pigments, nacres or fillers, which may be in dispersion in the aqueous continuous medium or the organic solvent medium of the composition.

However, these particles can have a tendency to sediment out over time, due to their density, which is greater than that of the continuous medium in which they are dispersed. This sedimentation may be reflected by a change in the macroscopic appearance of the composition, and for example, in the case of colored nail varnishes, by heterogeneity of the color of the varnish. This phenomenon may be accentuated when the temperature increases, such as when it exceeds 25° C.

In order to improve the stabilization of compositions, thickeners or gelling agents have been used; however, the incorporation of these agents in an amount required to stabilize the varnish may result in an increase in the viscosity, and thus a thickening (increase in consistency) of the product, which may be reflected by difficulties for the user in application to the nail.

Therefore, there is a need for a nail varnish composition that can have good stability and good color homogeneity over time, such as at temperatures above 25° C., and also a consistency that can allow the user easy application of the varnish to the nails.

The present inventor has found, surprisingly, that the use of a particular water-soluble or water-dispersible polymer can make it possible to obtain a composition that can have a satisfactory consistency over time. For example, this polymer is such that the viscosity of the composition comprising it increases when it is subjected to a temperature above the gel point of the polymer, in contrast with the water-based nail varnishes of the prior art, whose viscosity often may decrease when they are subjected to an increase in temperature.

Accordingly, one aspect of the present disclosure is a nail varnish composition comprising, in a cosmetically acceptable aqueous medium, at least one polymer chosen from water-soluble and water-dispersible polymers, the at least one polymer being such that the viscosity of the composition increases when it is subjected to a temperature above the gel point of the polymer.

For instance, the polymer can be such that, when the composition is subjected to a temperature of greater than or equal to the gel point of the polymer +20° C., the viscosity of the composition increases by at least 10%, relative to its viscosity measured at the gel point of the polymer, for a shear rate equal to 10 s⁻¹.

The polymer comprises, for example, water-soluble or water-dispersible units, and units with an LCST (Lower Critical Solution Temperature), wherein the units with an LCST have, in water, a solution temperature (or cloud point) ranging from 5 C to 40° C., where the units with an LCST are present in an amount of 1% by weight.

One aspect of the present disclosure is thus also a nail varnish composition comprising, in a cosmetically acceptable aqueous medium, at least one polymer chosen from water-soluble and water-dispersible polymers comprising water-soluble or water-dispersible units and units with an LCST, wherein the units with an LCST have a solution temperature in water (or cloud point) ranging from 5 to 40° C., wherein the units with an LCST are present in an amount of 1% by weight.

Another aspect of the present disclosure is also a non-therapeutic cosmetic process for making up or caring for the nails, comprising the application to the nails of at least one coat of the composition.

Still another aspect of the present disclosure is the use of at least one polymer chosen from water-soluble and water-dispersible polymers, comprising water-soluble or water-dispersible units, and units with an LCST, wherein the units with an LCST have a solution temperature in water ranging from 5 C to 40° C. and are present in an amount by weight of 1%, in a nail varnish composition comprising a cosmetically acceptable aqueous medium, to give the nail varnish composition color homogeneity over time, such as at temperatures above 25° C.

As used herein, the term “cosmetically acceptable aqueous medium” is understood to mean an aqueous medium that is compatible with the nails.

As used herein, the term “color homogeneity” is understood to mean a uniformity of the color of the composition from the bottom to the top of the container containing it, this characteristic being evaluated by macroscopic observation by the naked eye.

The nail varnish composition according to the present disclosure comprises at least one polymer chosen from water-soluble and water-dispersible polymers, wherein the at least one polymer comprises water-soluble or water-dispersible units, and units with an LCST, wherein the units with an LCST have a solution temperature in water ranging from 5 C to 40° C. and are present in an amount by weight of 1%.

As discussed below, the polymers disclosed herein have, in an aqueous phase, gelling properties stimulated by an increase in temperature, which can allow them to provide color homogeneity to the nail varnish compositions, yet without leading to formulations that are of very thick consistency and difficult to apply at room temperature (25° C.).

Water-soluble or water-dispersible polymers comprising water-soluble or water-dispersible units, and units with an LCST, have been described, for example, in documents such as: D. HOURDET et al., Polymer, 1994, vol. 35, No. 12, pages 2624 to 2630 [1]; F. L'ALLORET et al., Coll. Polym. Sci., 1995, vol. 273, No. 12, pages 1163-1173 [2]; F. L'ALLORET et al., Revue de l'Institut Frangais du Pétrole [Review of the French Petroleum Institute], 1997, vol. 52, No. 2, pages 117-128 [3]; and European Patent Nos. EP-A-0 583 814 [4] and EP-A-0 629 649 [5].

Thus, as described in these documents, the water-soluble or water-dispersible polymers comprise water-soluble or water-dispersible units, and units with an LCST that have a lower critical solution temperature in water. The units with an LCST are units whose solubility in water is modified above a certain temperature. They are units that have a heat-induced solution temperature (or cloud point) that defines their region of solubility in water. The minimum solution temperature obtained as a function of the polymer concentration is referred to as the “LCST” (Lower Critical Solution Temperature). For each polymer concentration, a heat-induced solution temperature is observed; it is higher than the LCST, which is the minimum point on the curve. Below this temperature, the polymer is soluble in water; above this temperature, the polymer loses its solubility in water.

The solution temperatures are determined by UV-visible spectroscopy by measuring, at a wavelength equal to 500 nm, the absorbance of aqueous solutions comprising 1% by weight of the units with an LCST. The solution temperature is identified at the temperature at which the absorbance of the solution is equal to 2.

According to the present disclosure, the at least one polymer comprised in the composition is one whose units with an LCST have a solution temperature ranging from 5 C to 40° C. in water, and are present in an amount by weight of 1%.

Thus, the polymers as disclosed herein have gelling properties in water caused by an increase in temperature. These properties are observed above the solution temperature of the units with an LCST, and are believed to be due to the combination of the units with an LCST within hydrophobic microdomains, thus forming crosslinking nodes between the main chains. This gelling power is reversible as a function of the temperature, in accordance with the thermodynamic origin of the physical crosslinking process involved.

The polymers as disclosed herein can make it possible to obtain nail varnish compositions whose consistency range at room temperature (25° C.) is broad in that the polymers can be fluid or gelled of greater or lesser consistency, and still conserve their color homogeneity when at a temperature above room temperature. The consistency or texture of these compositions at 25° C. may be adjusted as desired by introducing another gelling agent such as associative polyurethanes (for instance, Serad FX 1100 supplied by the company Servo) or hydrophilic clays (for instance, Laponite XLS supplied by the company Rockwood).

The gelling properties of the water-soluble or water-dispersible polymers comprising units with an LCST are observed when the concentration is sufficient to allow interactions between units with an LCST borne by different macromolecules. The minimum concentration required, known as the critical aggregation concentration or CAC, is evaluated by rheological measurements: it is the concentration at which and above which the viscosity of an aqueous solution of the polymers becomes higher than the viscosity of an aqueous solution of the equivalent polymer not comprising units with an LCST.

Above the CAC, the polymers as disclosed herein have gelling properties when the temperature becomes higher than a critical value, known as the gel point or T_{gel}. According to the literature data, there is good agreement between the gel point and the solution temperature of the units with an LCST, under the same concentration conditions. The gel point of an aqueous solution of a polymer of the present disclosure is determined by rheological measurements: it is the temperature at which and above which the viscosity of the polymer solution becomes higher than the viscosity of a solution of the equivalent polymer not comprising units with an LCST.

The gel points are determined in the following manner. It is considered that the gel point has been reached when the difference between the viscosity of the polymer solution and the viscosity of a solution of the equivalent polymer not comprising units with an LCST is greater than 5%.

The viscosity of the composition is measured using a Haake RS150 rheometer equipped with a 3.5 cm/2° or 6 cm/2° cone/plate geometry and a temperature control system. The viscosity measurements are performed in the flow mode by imposing a shear rate equal to 10⁻¹ s⁻¹.

According to the present disclosure, for example, polymers with a gel point ranging from 5 C to 50° C., for instance from 15° C. to 40° C., in an amount by weight in water of 2% can be used.

The polymers used in the context of the present disclosure can be in the form of block polymers comprising water-soluble or water-dispersible units and alternating units with an LCST, or in the form of grafted polymers comprising an optionally crosslinked skeleton formed from water-soluble or water-dispersible units and bearing grafts comprising units with an LCST, or vice-versa.

a) Water-soluble or Water-dispersible Units

The water-soluble or water-dispersible units of these polymers according to the present disclosure can be, for instance, units that are soluble in water, at a temperature ranging from 5° C. to 80° C., in an amount of at least 10 g/L, such as at least 20 g/L.

However, it is also possible to use, as the water-soluble or water-dispersible units, units not necessarily having the solubility mentioned above, but in aqueous solution at 1% by weight, at a temperature ranging from 5° C. to 80° C., which allow the production of a macroscopically homogeneous and transparent solution, i.e. a solution having a maximum light transmittance value, irrespective of the wavelength, ranging from 400 nm to 800 nm, through a sample 1 cm thick, of at least 85%, such as at least 90%.

The water-soluble or water-dispersible units do not have a heat-induced solution temperature of LCST type.

The water-soluble or water-dispersible units may be obtained by free-radical polymerization of vinyl monomers, or by polycondensation, or alternatively may comprise existing natural polymers or modified natural polymers.

Non-limiting examples that may be mentioned include the following water-soluble monomers and the salts thereof, which are capable of being used to form, totally or partially, by polymerization, the water-soluble or water-dispersible units, alone or as a mixture:
(meth)acrylic acid;
vinylsulfonic acid;
(meth)allylsulfonic acid;
vinylphosphonic acid;
methylvinylimidazolium chloride;
(meth)acrylamide;
2-vinylpyridine or 4-vinylpyridine;
maleic acid and anhydride;
crotonic acid;
itaconic acid;
vinyl alcohol of formula CH₂═CHOH;
N-vinyllactams comprising a cyclic alkyl group of 4 to 9 carbon atoms, such as N-vinylpyrrolidone, N-vinylcaprolactam and N-butyrolactam;
water-soluble styrene derivatives, such as styrene sulfonate;
dimethyldiallylammonium chloride;
N-vinylacetamide and N-methyl-N-vinylacetamide;
N-vinylformamide and N-methyl-N-vinylformamide;
vinyl monomers of formula (I): 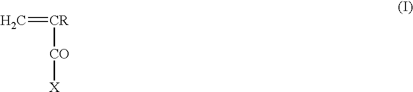
   wherein:

R is chosen from H, —CH₃, —C₂H₅ and —C₃H₇ groups, and

X is chosen from:
alkyl oxides of —OR′ type in which R′ is chosen from linear and branched, saturated and unsaturated hydrocarbon-based radicals comprising from 1 to 6 carbon atoms, substituted with at least one entity chosen from halogen atoms (such as iodine, bromine, chlorine or fluorine); sulfonic groups (—SO₃⁻), sulfate groups (—SO₄⁻), phosphate groups (—PO₄H₂₂⁻); hydroxyl groups (—OH); ether groups; primary amine groups (—NH₂); secondary amine groups (—NHR₁), tertiary amine groups (—NR₁R₂) and quaternary amine groups (—N⁺R₁R₂R₃) wherein R₁, R₂ and R₃, which may be identical or different, are chosen from linear and branched, saturated and unsaturated hydrocarbon-based radicals comprising from 1 to 6 carbon atoms, with the proviso that the sum of the carbon atoms of R′+R₁+R₂+R₃ does not exceed 7; and
—NH₂, —NHR₄ and —NR₄R₅ groups wherein R₄ and R₅, which may be identical or different, are chosen from linear and branched, saturated and unsaturated hydrocarbon-based radicals comprising from 1 to 6 carbon atoms, with the proviso that the total number of carbon atoms of R₄+R₅ does not exceed 7, and wherein the R₄ and R₅ groups are substituted with at least one entity chosen from halogen atoms (such as iodine, bromine, chlorine or fluorine); hydroxyl groups (—OH); ether groups ; sulfonic groups (—SO₃⁻); sulfate groups (—SO₄⁻); phosphate groups (—PO₄H₂⁻); primary amine groups (—NH₂); secondary amine groups (—NHR₁), tertiary amine groups (—NR₁R₂) and/or quaternary amine groups (—N⁺R₁R₂R₃) wherein R₁, R₂ and R₃, which may be identical or different, are chosen from linear and branched, saturated and unsaturated hydrocarbon-based radicals comprising from 1 to 6 carbon atoms, with the proviso that the sum of the carbon atoms of R₄+R₅+R₁+R₂+R₃ does not exceed 7.

Non-limiting examples of water-soluble monomers of formula (I) that may be mentioned include glycidyl (meth)acrylate, hydroxyethyl methacrylate, ethylene glycol (meth)acrylates, diethylene glycol, polyalkylene glycol, N,N-dimethylacrylamide, acrylamido-2-methylpropanesulfonic acid (AMPS), (meth)acrylamidopropyltrimethylammonium chloride (APTAC and MAPTAC) and quaternized dimethylaminoethyl (DAMEMA).

Among the monomers that may be used to constitute, totally or partially, the water-soluble or water-dispersible units by polymerization, non-limiting mention may also be made of hydrophobic monomers, the monomers being copolymerized with at least one water-soluble monomer in an amount such that the resulting units are water-soluble or water-dispersible, wherein the hydrophobic monomers are chosen from the following monomers:
styrene and its derivatives such as 4-butylstyrene, α-methylstyrene and vinyltoluene;
vinyl acetate of formula CH₂═CH—OCOCH₃;
vinyl ethers of formula CH₂═CHOR₆, in which R₆ is chosen from linear and branched, saturated and unsaturated hydrocarbon-based radicals comprising from 1 to 6 carbon atoms;
acrylonitrile;
caprolactone;
vinyl chloride and vinylidene chloride;
silicone derivatives such as methacryloxypropyltris(trimethylsiloxy)silane and silicone-based methacrylamides;
hydrophobic vinyl monomers chosen from those of formula (II): 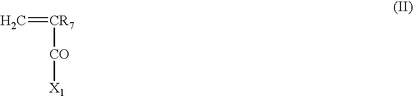
   wherein:

R₇ is chosen from H, —CH₃, —C₂H₅ and C₃H₇ groups,

X₁ is chosen from:
alkyl oxides of the type —OR₈ wherein R₈ is chosen from linear and branched, saturated and unsaturated hydrocarbon-based radicals comprising from 1 to 8 carbon atoms; and
NH₂, —NHR₉ and —NR₉R₁₀ groups wherein R₉ and R₁₀, which may be identical or different, are chosen from linear and branched, saturated and unsaturated hydrocarbon-based radicals comprising from 1 to 6 carbon atoms, with the proviso that the total number of carbon atoms in R₉+R₁₀ does not exceed 6.

Non-limiting examples of such monomers that may be mentioned include methyl methacrylate, ethyl methacrylate, n-butyl methacrylate, tert-butyl methacrylate and cyclohexyl acrylate.

The water-soluble or water-dispersible units may be neutralized, where appropriate, totally or partially, with a mineral or organic base chosen from salts of sodium, ammonium, lithium, calcium, magnesium, ammonium substituted with 1 to 4 alkyl groups bearing from 1 to 15 carbon atoms, or alternatively from monoethanolamine, diethanolamine, triethanolamine, aminoethylpropanediol, N-methylglucamine, basic amino acids such as arginine and lysine, and mixtures thereof.

It may also be noted that the skeleton of the grafted polymers can be crosslinked by the action of at least one crosslinking agent chosen from the polyolefinically unsaturated compounds commonly used for crosslinking polymers obtained by free-radical polymerization. Examples of such agents that may be mentioned include divinylbenzene, diallyl ether, dipropylene glycol diallyl ether, polyglycol diallyl ethers, triethylene glycol divinyl ether, hydroquinone diallyl ether, tetrallyl-oxethanoyl, polyfunctional alcohol allyl or vinyl ether derivatives, tetraethylene glycol diacrylate, triallylamine, trimethylolpropane diallyl ether, tetraallyloxyethane, methylenebisacrylamide, allyl ethers of alcohols of the sugar series, allyl methacrylate and trimethylolpropane triacrylate (TMPTA), and mixtures thereof.

For example, a non-crosslinked grafted polymer skeleton may be used.

Among the polycondensates and the natural polymers and/or modified natural polymers capable of constituting, totally or partially, the water-soluble or water-dispersible units, non-limiting mention may be made of:
water-soluble polyurethanes,
xanthan gum, such as the products sold under the names Keltrol T and Keltrol SF by Kelco, or Rhodigel SM and Rhodigel 200 from Rhodia;
alginates (Kelcosol from Monsanto) and derivatives thereof such as propylene glycol alginate (Kelcoloid LVF from Kelco);
cellulose derivatives, for instance carboxymethylcellulose (Aquasorb A500 from Hercules), hydroxypropylcellulose, hydroxyethylcellulose and quaternized hydroxyethylcellulose;
galactomannans and derivatives thereof, such as konjac gum, guar gum, hydroxypropyl guar, hydroxypropyl guar modified with sodium methylcarboxylate groups (Jaguar XC97-1 from Rhodia) and guar hydroxypropyltrimethylammonium chloride; and
polyethyleneimine.

The water-soluble or water-dispersible units can have, for example a molar mass ranging from 1,000 g/mol to 10,000,000 g/mol when they constitute the water-soluble skeleton of a grafted polymer. The water-soluble or water-dispersible units can have, for example, a molar mass ranging from 500 g/mol to 500,000 g/mol when they constitute a block of a multiblock polymer or when they constitute the grafts of a grafted polymer.

b) Units with an LCST

The units with an LCST in the polymers used in the present disclosure may be defined as units whose water solubility is modified above a certain temperature. They are units with a heat-induced solution temperature (or cloud point) defining their region of solubility in water. As discussed above, the minimum solution temperature obtained as a function of the polymer concentration is referred to as the “LCST” (Lower Critical Solution Temperature). For each polymer concentration, a heat-induced solution temperature is observed; it is higher than the LCST, which is the minimum point of the curve. Below this temperature, the polymer is soluble in water; above this temperature, the polymer loses its solubility in water.

As used herein, the expression “soluble in water at a temperature T” is understood to mean that the units have a solubility at T of at least 1 g/l, for instance, at least 2 g/l.

The measurement of the solution temperature can be performed visually. For example, the temperature at which the cloud point of the aqueous solution appears is determined visually, since, at this temperature, opacification of the solution, or loss of transparency, takes place.

In general, the solution temperature can be determined by UV-visible spectroscopy by measuring, at a wavelength equal to 500 nm, the absorbance of an aqueous solution comprising 1% by weight of the units with an LCST. The solution temperature is identified as the temperature at which the absorbance of the solution is equal to 2.

The units with an LCST in the polymers used in the present disclosure can comprise at least one of the following polymers:
polyethers such as polyethylene oxide (PEO), polypropylene oxide (PPO) and random copolymers of ethylene oxide (EO) and of propylene oxide (PO),
polyvinyl methyl ether,
polymeric and copolymeric N-substituted acrylamide derivatives with an LCST, such as poly-N-isopropylacrylamide and poly-N-ethylacrylamide, and
polyvinylcaprolactam and vinylcaprolactam copolymers.

For example, the units with an LCST comprise random copolymers of ethylene oxide (EO) and of propylene oxide (PO), represented by the formula:
(EO)ₘ(PO)ₙ
wherein m is an integer ranging from 1 to 40, such as from 2 to 20, and n is an integer ranging from 10 to 60, such as from 20 to 50.

The molar mass of these units with an LCST can range from 500 g/mol to 5300 g/mol, for instance, from 1500 g/mol to 4000 g/mol.

Without being bound by theory, it has been found that the random distribution of the EO and PO units is reflected by the existence of a Lower Critical Solution Temperature, above which a macroscopic phase separation is observed. This behavior is different from that of the block EO PO copolymers, which undergo micellization above a critical temperature known as the micellization temperature (microscopic aggregation).

The units with an LCST for example, can thus be in the form of amino, such as monoamino, diamino or triamino, random copolymers of ethylene oxide and of propylene oxide. Among the commercially available units with an LCST that may be used, non-limiting mention may be made of the copolymers sold under the name Jeffamine by Huntsman, and for instance, Jeffamine XTJ-507 (M-2005), Jeffamine D-2000 and Jeffamine XTJ-509 (or T-3000).

The units with an LCST may also be in the form of random EO/PO copolymers comprising OH end groups, such as those sold under the name Polyglycols P41 and B11 by Clariant.

For example, it is possible according to the present disclosure to use polymers comprising units with an LCST, such as polymeric and copolymeric N-substituted acrylamide derivatives with an LCST.

Non-limiting examples of N-substituted acrylamide derivatives that may be mentioned include poly-N-isopropylacrylamide, poly-N-ethylacrylamide and copolymers of N-isopropylacrylamide or of N-ethylacrylamide and of vinyl monomers chosen from those of formulae (I) and (II) described above, or of a monomer chosen from (meth)acrylic acid, vinylsulfonic acid, (meth)allylsulfonic acid, maleic acid and anhydride, vinylphosphonic acid, crotonic acid, itaconic acid, (meth)acrylamide, vinylpyridine, vinyl alcohol, N-vinyllactams such as N-vinylpyrrolidone, styrene and its derivatives, dimethyldiallylammonium chloride, N-vinylacetamide, N-methyl-N-vinylacetamide, N-vinylformamide, N-methylvinylformamide, vinyl ethers, vinyl acetate, acrylonitrile, caprolactone, vinyl chloride and vinylidene chloride.

For example, the molar mass of the N-substituted acrylamide polymers and copolymers can range from 1,000 g/mol to 500,000 g/mol.

These polymers can be synthesized by free-radical polymerization using a pair of initiators such as aminoethanethiol hydrochloride in the presence of potassium persulfate, so as to obtain oligomers with an amino reactive end group.

Non-limiting examples of N-vinylcaprolactam copolymers that may be mentioned include copolymers of N-vinylcaprolactam and of vinyl monomers chosen from those of formulae (I) and (II) given above, or of monomers chosen from (meth)acrylic acid, vinylsulfonic acid, (meth)allylsulfonic acid, maleic acid, maleic anhydride, vinylphosphonic acid, crotonic acid, itaconic acid, (meth)acrylamide, vinylpyridine, vinyl alcohol, N-vinyllactams such as N-vinylpyrrolidone, styrene and its derivatives, dimethyldiallylammonium chloride, N-vinylacetamide, N-methyl-N-vinylacetamide, N-vinylformamide, N-methylvinylformamide, vinyl acetate, vinyl ethers, acrylonitrile, caprolactone, vinyl chloride and vinylidene chloride.

The molar mass of these polymers can range, for example, from 1,000 g/mol to 500,000 g/mol.

The polymers can be synthesized by free-radical polymerization using a pair of initiators such as aminoethanethiol hydrochloride in the presence of azobisisobutyronitrile, so as to obtain oligomers with an amino reactive end group.

The units with an LCST can be present in the final polymer in an amount, for example, ranging from 5% to 70%, for instance from 20% to 65%, such as from 30% to 60% by weight, relative to the weight of the final polymer.

As defined above, the heat-induced solution temperature of the units with an LCST ranges from 5° C. to 40° C., such as from 10° C. to 35° C., for an amount by weight of 1% in water of the units with an LCST.

The polymers used in the context of the present disclosure can be readily prepared by a person skilled in the art by various methods, such as grafting, copolymerization, coupling or living polymerization processes.

For example, when the final polymer is in the form of a grafted polymer having, for instance, a water-soluble skeleton with side units with an LCST, it is possible to prepare it by grafting the units with an LCST comprising at least one reactive end group, for example an amino group, onto a water-soluble polymer forming the skeleton, the skeleton bearing at least 10 mol % of reactive groups such as carboxylic acid functional groups. The reaction may be performed in the presence of a carbodiimide such as dicyclohexylcarbodiimide or 1-(3-dimethylaminopropyl)-3-ethylcarbodiimide hydrochloride, in a solvent such as N-methylpyrrolidone or water.

Another possibility for preparing grafted polymers comprises copolymerizing, for example, a macromonomer with an LCST (unit described previously with an unsaturated end group) and a water-soluble vinyl monomer such as acrylic acid or vinyl monomers of formula (I) described above.

When the final polymer is in the form of a block polymer, it can be prepared by coupling between water-soluble or water-dispersible units and units with an LCST having complementary reactive sites at each end.

It is also possible to prepare such polymers by living polymerization of anionic or cationic type, or by controlled free-radical polymerization. The latter synthetic process can be performed by various methods, for instance the atom-transfer method (Atom Transfer Radical Polymerization or ATRP), the method with free radicals such as nitroxides, or via the method by reversible chain transfer with addition-fragmentation (Radical Addition-Fragmentation Chain Transfer) such as the MADIX process (Macromolecular Design via the Interchange of Xanthate). These processes can be used to obtain the water-soluble blocks and the blocks with an LCST of the polymers used in the context of the present disclosure. They may also be used to synthesize only one of the two types of polymer block used according to the present disclosure, the other block being introduced into the final polymer via the initiator used, or alternatively by coupling reaction between the water-soluble blocks and the blocks with an LCST.

The polymer can be present in the composition according to the present disclosure in a solids amount ranging from 0.01% to 20% by weight, relative to the total weight of the composition, such as from 0.1% to 15% by weight, relative to the total weight of the composition

c) Physiologically Acceptable Aqueous Medium

The aqueous medium of the composition according to the present disclosure can consist essentially of water (for example, the aqueous medium can be water) or can comprise a mixture of water and at least one water-miscible solvent, for instance lower monoalcohols comprising from 1 to 5 carbon atoms, such as ethanol, glycols comprising from 2 to 8 carbon atoms, C₃-C₄ ketones and C₂-C₄ aldehydes.

The aqueous medium of the composition can be present in an amount ranging from 5% to 95% by weight, such as from 20% to 80% by weight, relative to the total weight of the composition.

The composition according to the present disclosure may comprise, besides the polymer comprising units with an LCST, at least one thickener, for example, an aqueous-medium thickener, such as hydrophilic clays, for instance hectorites, bentonites, for instance Laponite XLS sold by the company Rockwood, associative thickeners such as associative polyurethanes, for instance Serad FX sold by the company Servo, associative acrylic polymers, water-soluble cellulose-based thickeners such as hydroxyethylcellulose, and natural gums, such as xanthan gum.

The at least one thickener, when present, may be present in an amount ranging from 0.05% to 5% by weight, relative to the total weight of the composition.

The composition may also comprise at least one film-forming polymer (as an additional polymer) in a solids amount (or active material amount) ranging from 5% to 70% by weight, such as from 10% to 60% by weight, for instance, from 20% to 50% by weight, relative to the total weight of the composition. As used herein, the term “film-forming polymer” is understood to mean a polymer that is capable, by itself or in the presence of a possible plasticizer, of forming an isolable film. The at least one film-forming polymer, when present, can be dissolved or dispersed in the form of particles in the cosmetically acceptable medium of the composition.

The at least one film-forming polymer may be chosen from free-radical polymers obtained from monomers comprising at least one unsaturation, polycondensates and polymers of natural origin. For example, the film-forming polymer may be chosen vinyl and acrylic polymers, polyurethanes, polyesters, alkyd resins, epoxyester resins, cellulose-based polymers, such as nitrocellulose, cellulose esters, for instance cellulose acetate, cellulose acetopropionate or cellulose acetobutyrate, resins resulting from the condensation of formaldehyde with an arylsulfonamide, and mixtures thereof.

The film-forming polymer may be present in the form of particles in dispersion in the aqueous medium of the composition, thus forming a latex or pseudolatex. The polymer particles can have a size ranging from 10 nm to 200 nm, for instance, ranging from 20 nm to 150 nm, such as from 50 nm to 100 nm.

Among the film-forming polymers that may be used in the composition according to the present disclosure, non-limiting mention may be made of polyurethanes, for example anionic polyurethanes, polyester-polyurethanes, polyether-polyurethanes, free-radical polymers, for instance of acrylic, styrene-acrylic and/or vinylic type, polyesters and alkyd resins, alone or as a mixture.

The at least one film-forming polymer in aqueous dispersion can be chosen from aqueous dispersions of acrylic, styrene/acrylic and vinylic polymers and/or copolymers, being present in a solids amount ranging from 30% to 50% by weight.

The acrylic film-forming polymers that may be used according to the present disclosure can result from the polymerization of at least one ethylenically unsaturated monomer chosen from ethylenic-carboxylic acids, esters thereof and amides thereof. Non-limiting examples of ethylenic unsaturated carboxylic acids that may be used include acrylic acid, methacrylic acid, crotonic acid, maleic acid and itaconic acid. For example, (meth)acrylic acid and crotonic acid may be used, and in at least one embodiment of the present disclosure, (meth)acrylic acid is used. The esters of these carboxylic acids can be chosen from the esters of (meth)acrylic acid (also known as (meth)acrylates), such as alkyl (meth)acrylates, for instance of a C₁-C₃₀, such as a C₁-C₂₀ alkyl, aryl(meth)acrylates, for example, of a C₆-C₁₀ aryl, hydroxyalkyl (meth)acrylates, for instance of a C₂-C₆ hydroxyalkyl. Among the alkyl (meth)acrylates that may be used, non-limiting mention may be made of methyl methacrylate, ethyl methacrylate, butyl methacrylate, isobutyl methacrylate, 2-ethylhexyl methacrylate, lauryl methacrylate and cyclohexyl methacrylate. Among the hydroxyalkyl (meth)acrylates that may be used, non-limiting mention may be made of hydroxyethyl acrylate, 2-hydroxypropyl acrylate, hydroxyethyl methacrylate and 2-hydroxypropyl methacrylate. Needless to say, it is possible to use a mixture of these monomers. In at least one embodiment of the present disclosure, the (meth)acrylic acid esters are chosen from alkyl (meth)acrylates. According to the present disclosure, the alkyl group may be either fluorinated or perfluorinated, i.e., some or all of the hydrogen atoms of the alkyl group are replaced with fluorine atoms.

Among the amides of the carboxylic acids that may be used, non-limiting mention may be made of, for example, (meth)acrylamides, such as N-alkyl(meth)acrylamides, for instance of a C₂-C₁₂ alkyl. Among the N-alkyl(meth)acrylamides that may be used, non-limiting mention may be made of N-ethylacrylamide, N-butylacrylamide, N-octylacrylamide and N-undecylacrylamide.

The acrylic film-forming polymer that may be used according to the present disclosure may comprise, in addition to the monomers mentioned above, at least one styrene monomer, such as styrene or a-methylstyrene.

Among the acrylic polymers in aqueous dispersion that may be used, non-limiting mention may be made of the acrylic polymers dispersions sold under the names Neocryl XK-90®, Neocryl A-1070®, Neocryl A-1090®, Neocryl BT-62®, Neocryl A-1079®, Neocryl A-523® and Neocryl XK63 by the company Avecia Resins, Dow Latex 432® by the company Dow Chemical, Joncryl77 or Joncryl90 by the company Johnson, Luhydran A848S by the company BASF, and Syntran 5190 by the company Interpolymer.

The at least one film-forming polymer in aqueous dispersion can be chosen from aqueous dispersions of anionic polyurethane, of polyester-polyurethane, of polyether-polyurethane, and a mixture thereof, which can, for instance, have a solids amount ranging from 20% to 40%. The aqueous polyurethane dispersion can be, for example, an aqueous dispersion of anionic polyurethane, of polyester-polyurethane and/or of polyether-polyurethane, alone or as a mixture, which can have a solids amount ranging from 20% to 40%.

Among the aqueous polyurethane dispersions that may be used, non-limiting mention may be made of the polyester-polyurethanes sold under the names “Avalure UR-405®,” “Avalure UR-410®,” “Avalure UR-425®” and “Sancure 2060®” by the company Goodrich, the polyether-polyurethanes sold under the names “Sancure 878®” by the company Goodrich, “Neorez R-970®” by the company Avecia, or the aqueous polyurethane dispersions obtained by polycondensation of tetramethylxylylene diisocyanate, such as “Cydrothane HP 1035,” “Cydrothane HP 4033,” “Cydrothane HP 5035” and “Cydrothane HP 6000” from the company Cytec or the dispersions sold under the name “IW/01.1, IW/019.1, IW/028.1” by the company UCB.

To improve the film-forming properties of the nail varnish composition, at least one auxiliary film-forming agent may also be present in the composition. The at least one auxiliary film-forming agent can be chosen from any compound known to those skilled in the art as being capable of satisfying the desired function, and can be chosen from, for example, plasticizers and coalescers, and a mixture thereof.

Among the plasticizers that may be used as disclosed herein, non-limiting mention may be made of, alone or as a mixture, known plasticizers, such as:
glycols and derivatives thereof, such as diethylene glycol ethyl ether, diethylene glycol methyl ether, diethylene glycol butyl ether or diethylene glycol hexyl ether, ethylene glycol ethyl ether, ethylene glycol butyl ether or ethylene glycol hexyl ether;
glycol esters;
propylene glycol derivatives, for instance propylene glycol phenyl ether, propylene glycol diacetate, dipropylene glycol butyl ether, tripropylene glycol butyl ether, propylene glycol methyl ether, dipropylene glycol ethyl ether, tripropylene glycol methyl ether, diethylene glycol methyl ether and propylene glycol butyl ether;
acid esters, for instance carboxylic acid esters, such as citrates, phthalates, adipates, carbonates, tartrates, phosphates and sebacates;
oxyethenylated derivatives, such as oxyethenylated oils, for instance plant oils, such as castor oil; and
mixtures thereof.

The amount of total plasticizer present can be chosen by a person skilled in the art on the basis of his general knowledge, so as to obtain a composition having cosmetically acceptable properties. The at least one plasticizer may be present in a total amount ranging, for example, from 0.1% to 15% by weight, such as from 0.5% to 10% by weight relative to the total weight of the composition.

The composition may also comprise at least one dyestuff, which can be chosen from pulverulent compounds and/or water-soluble dyes. The dyestuff can be present in an amount ranging from 0.01% to 50% by weight, for instance from 0.05% to 30% by weight, such as from 0.1% to 20% by weight, relative to the total weight of the composition.

The pulverulent compounds may be chosen from the pigments and/or nacres and/or flakes usually used in nail varnishes.

The pigments may be white or colored, and mineral and/or organic. Among the mineral pigments that may be used as disclosed herein, non-limiting mention may be made of titanium dioxide, optionally surface-treated, zirconium oxide or cerium oxide, and also iron oxide, chromium oxide, manganese violet, ultramarine blue, chromium hydrate and ferric blue, and metallic pigments, for instance aluminium, copper or bronze. Among the organic pigments that may be used, non-limiting mention may be made of carbon black, pigments of D & C type and lakes based on cochineal carmine, barium, strontium, calcium or aluminium, and guanine.

Non-limiting mention may also be made of pigments with an effect, such as particles comprising a natural or synthetic, organic or mineral substrate, for example glass, acrylic resins, polyester, polyurethane, polyethylene terephthalate, ceramics or aluminas, the said substrate being uncoated or coated with metal substances, for instance aluminium, gold, copper or bronze, or with metal oxides, for instance titanium dioxide, iron oxide or chromium oxide, mineral or organic pigments and mixtures thereof.

The nacreous pigments can be chosen from white nacreous pigments such as mica coated with titanium or with bismuth oxychloride, colored nacreous pigments such as titanium mica coated with iron oxides, titanium mica coated for instance, with ferric blue or chromium oxide, titanium mica coated with an organic pigment of the abovementioned type and also fluorophlogopite with iron oxides.

Pigments with goniochromatic properties, such a liquid-crystal pigments or multilayer pigments, may also be used.

The water-soluble dyes can be, for example, beetroot juice or methylene blue.

The composition according to the present disclosure may additionally comprise at least one filler, for example in an amount ranging from 0.01% to 50% by weight, such as ranging from 0.01% to 30% by weight, relative to the total weight of the composition. As used herein, the term “fillers” is understood to mean colorless or white, mineral or synthetic particles of any shape, which are insoluble in the medium of the composition, irrespective of the temperature at which the composition is manufactured. These fillers serve, for instance, to modify the rheology or the texture of the composition.

The fillers may be mineral or organic in any form, platelet-shaped, spherical or oblong, irrespective of the crystallographic form (for example leaflet, cubic, hexagonal, orthorhombic, etc.). Among the fillers that may be used as disclosed herein, non-limiting mention may be made of talc, mica, silica, kaolin, polyamide (Nylon®) powders (Orgasol® from Atochem), poly-β-alanine powder and polyethylene powder, powders of tetrafluoroethylene polymers (Teflon®), lauroyllysine, starch, boron nitride, hollow polymer microspheres such as those of polyvinylidene chloride/acrylonitrile, for instance Exapancel® (Nobel Industrie) or acrylic acid copolymers (Polytrap® from the company Dow Corning) and silicone resin microbeads (for example Tospearls® from Toshiba), elastomeric polyorganosiloxane particles, precipitated calcium carbonate, magnesium carbonate, magnesium hydrocarbonate, hydroxyapatite, hollow silica microspheres (Silica Beads® from Maprecos), glass or ceramic microcapsules, and metal soaps derived from organic carboxylic acids comprising from 8 to 22 carbon atoms, such as from 12 to 18 carbon atoms, for example zinc, magnesium or lithium stearate, zinc laurate or magnesium myristate.

The composition may also comprise at least one adjuvant or additive commonly used in cosmetic compositions and known to those skilled in the art as being able to be incorporated into a nail varnish composition.

Such adjuvants or additives may be chosen from coalescers, thickeners (such as hydrophilic clays, associative polyurethanes, associative polyacrylic acids, cellulose and cellulose derivatives, gums, and mixtures thereof), preserving agents, fragrances, oils, waxes, surfactants, antioxidants, free-radical scavengers, spreading agents, wetting agents, dispersants, antifoams, neutralizers, stabilizers, active agents chosen from essential oils, organic and mineral UV/solar screening agents, fragrances, moisturizers, vitamins, proteins, ceramides, plant extracts, etc., and mixtures thereof.

In at least one embodiment, the nail varnish composition according to the present disclosure may be free of an oily phase comprising at least one oil of the type such as alkanes, esters, triglycerides, ethers, silicone oils and fluoro oils.

Needless to say, a person skilled in the art will take care to select the at least one optional additional compound(s) and/or the amount thereof such that the properties of the composition according to the present disclosure are not, or are not substantially, adversely affected by the envisaged addition.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements.

The following examples are intended to illustrate the present disclosure in a non-limiting manner. Unless otherwise mentioned, the amounts indicated are expressed in grams.

### EXAMPLES

### Example 1

### Preparation of a Polymer Containing Water-soluble Units and Units With an LCST

A polymer comprising a water-soluble skeleton of polyacrylic acid (PAA) 450,000 g/mol bearing 3.9 mol % of side chains or grafts with an LCST, which are random copolymers of ethylene oxide (6) and of propylene oxide (39) (Jeffamine M-2005 of MW 2600) was prepared.

3 grams of polyacrylic acid of mean molar mass 450,000 g/mol (Aldrich) were dissolved in 220 ml of N-methylpyrrolidone in a 500 ml reactor equipped with a condenser, with stirring at 60° C. for 12 hours.

4.181 grams of random monoamino copolymer (EO)₆(PO)₃₉, of molar mass 2,600 g/mol having a cloud point, at a concentration of 1% by weight in water, of 16° C. (Jeffamine M-2005 from Huntsman) were dissolved in 50 ml of N-methylpyrrolidone with stirring, at 20° C., for 15 minutes. The solution obtained was added dropwise to the reaction medium comprising the polyacrylic acid, with vigorous stirring at 60° C.

2.158 grams of dicyclohexylcarbodiimide were dissolved in 30 ml of N-methylpyrrolidone with stirring at 20° C. for 15 minutes. The solution obtained was added dropwise to the reaction medium comprising the polyacrylic acid and the random monoamino (EO)₆(PO)₃₉ copolymer, with vigorous stirring at 60° C. The final mixture was stirred for 12 hours at 60° C.

The mixture was cooled to 20° C. and was then placed in a refrigerator at 4° C. for 24 hours. The crystals of dicyclohexylurea formed were removed by filtration of the reaction medium.

The polymer was then neutralized with 19 g of 35% sodium hydroxide (fourfold excess relative to the number of moles of acrylic acid), leading to its precipitation. After 12 hours at rest, the reaction medium was filtered to recover the precipitated polymer. This polymer was dried under vacuum at 35° C. for 24 hours.

13.55 grams of solid were recovered, and were dissolved in 2 litres of deionized water. This solution was ultrafiltered using a Millipore ultrafiltration system comprising a membrane with a cutoff threshold set at 10,000 daltons. The solution thus purified was freeze-dried to collect the polymer in solid form.

7.05 grams of polyacrylic acid (450,000 g/mol) grafted with 3.9 mol % of random monoamino (EO)₆(PO)₃₉ copolymer were obtained.

The units with an LCST were present in the final polymer in an amount by weight of 51%.

The polymer obtained was characterized by the molar mass of the water-soluble skeleton (polyacrylic acid), the chemical nature of the chains with an LCST, their mass proportion in the polymer and their molar mass, as follows:

| | | | Proportion: units | |
|---|---|---|---|---|
| | | | with an LCST in | Degree of |
| | Water-soluble | Grafts (units | the final polymer | grafting |
| | skeleton | with an LCST) | (by weight) | (mol %) |
| Polymer | polyacrylic | random | 51% | 3.9% |
| | acid: | (EO)₆(PO)₃₉ | | |
| | MW = 450 000 | Jeffamine | | |
| | | M-2005: | | |
| | | MW = 2600 | | |

The polymer had the following characteristics:

| Solution temperature of the grafts at 1% in water: | 23° C. |
|---|---|
| Gel point of the polymer at 2% in water | 28° C. |
| Critical aggregation concentration of the polymer in water | 0.9% |

The solution temperature of the Jeffamine units was determined by UV-visible spectroscopy by measuring, at a wavelength equal to 500 nm, the absorbance of an aqueous solution comprising 1% by weight of the units with an LCST. The solution temperature was identified as the temperature at which the absorbance of the solution was equal to 2.

The gel point of an aqueous solution of a polymer of the present disclosure was determined by Theological measurements: it is the temperature at which and above which the viscosity of the solution of the polymer becomes higher than the viscosity of a solution of the equivalent polymer not comprising units with an LCST.

The gel point of a 2% solution of polymer in water corresponded to the expected gel point when the difference between the viscosity of the polymer solution and the viscosity of a solution of the equivalent polymer not comprising units with an LCST was greater than 5%. The viscosity of the composition was measured using a Haake RS 150 rheometer equipped with a 3.5 cm/²° or 6 cm/²° cone/plate geometry and a temperature control system. The viscosity measurements were performed in flow mode by imposing a shear rate equal to 10⁻¹s⁻¹.

The critical aggregation concentration (CAC) of the polymer was determined in pure water by rheology. It was the concentration at which and above which the viscosity of an aqueous solution of the polymer under consideration becomes higher than the viscosity of a solution of the equivalent polymer not comprising units with an LCST. The viscosity measurement was performed using a Haake RS 150 rheometer equipped with a cone/plate geometry (35 mm, 2° C.) and a thermostatically regulated bath in order to control the temperature ranging from 5 to 80° C. The measurements were performed in flow mode at a shear rate of 10 s⁻¹, by varying the temperature from 15° C. to 50° C. at a rate of 0.5° C./minute.

### Examples 2 and 3

### White-Shade Nail Varnishes

Two white-shade nail varnishes were prepared:
The varnish of Example 2 according to the present disclosure comprised the gelling polymer of Example 1;

the nail varnish of Example 3 (comparative) did not comprise a polymer comprising water-soluble units and units with an LCST, and comprised an associative polyurethane (Serad FX 1100).

| | Example 2 | Example 3 |
|---|---|---|
| Constituents | (Inventive) | (Comparative) |
| Polymer of Example 1 | 0.5 | |
| Associative polyurethane sold under the | | 0.08 |
| name “Serad FX 1100” by the company | | |
| Servo Huls | | |
| Aqueous polyurethane dispersion at a | 30.9 (AM) | 30.9 (AM) |
| solids content of 35% by weight, sold | | |
| under the name “Cydrothane HP 6000” | | |
| by the company Cytec | | |
| White mineral dye sold under the name | 1.6 | 1.6 |
| “Blanc Covarine W9797” by the company | | |
| Wackherr | | |
| Diazolidinylurea | 0.39 | 0.39 |
| Perfluorohexyl betaine | 0.05 | 0.05 |
| Ethanol | 4.25 | 4.25 |
| Preserving agents | 0.54 | 0.54 |
| Demineralized water | qs 100 | qs 100 |

Evaluation of the Color Homogeneity

The varnishes were stored at 45° C. for two months:

The varnish of Example 2 had good color homogeneity over time: after having been placed at 45° C. (temperature above the gel point of the sodium polyacrylate polymer comprising Jeffamine grafts, which is 28° C.) for 2 months, it remained white from the top to the bottom of the bottle containing it.

The varnish of Example 3 showed colour heterogeneity (appearance of a translucent supernatant at the top of the bottle containing it).

The gelling power of the polymer was demonstrated by measuring the viscosity of the above nail varnish compositions.

The viscosity measurements were performed using a Haake RS 150 rheometer equipped with a cone/plate geometry (35 mm, 2°) and a thermostatically regulated path in order to control the temperature ranging from 4° C. to 80° C. The measurements were performed in the flow mode, at an imposed shear rate equal to 10 s⁻¹, by varying the temperature from 4° C. to 45° C. at a rate of 0.5° C./minute.

| | Example 2 | Example 3 |
|---|---|---|
| Viscosity at 20° C. (10 s⁻¹) | 0.08 Pa · s | 0.08 Pa · s |
| Viscosity at 35° C. (10 s⁻¹) | 0.15 Pa · s | 0.04 Pa · s |

The viscosity of the varnish of Example 2 increased when the temperature is higher than the gel point of the polymer.

The viscosity of Example 3, which does not comprise a polymer with an LCST, decreases when the temperature increases.

### Examples 4 and 5

### Pink-Shade Nail Varnishes

Two pink-shade nail varnishes were prepared:
The varnish of Example 4 according to the present disclosure comprised the gelling polymer of Example 1
The nail varnish of Example 5 (comparative) did not comprise a polymer comprising water-soluble units and units with an LCST, and comprised an associative polyurethane (Serad FX 1100).

The compositions are given in the following table:

| | Example 4 | Example 5 |
|---|---|---|
| Constituents | (Inventive) | Comparative |
| Polymer of Example 1 | 0.5 | |
| Associative polyurethane sold under the | | 0.08 |
| name “Serad FX 1100” by the company | | |
| Servo Huls | | |
| Aqueous polyurethane dispersion at a | 30.9 (AM) | 30.9 (AM) |
| solids content of 35% by weight, sold under | | |
| the name “Cydrothane HP 6000” by the | | |
| company Cytec | | |
| White mineral dye sold under the name | 0.8 | 0.8 |
| “Blanc Covarine W9797” by the company | | |
| Wackherr | | |
| Red organic dye sold under the name | 1.2 | 1.2 |
| “Rouge Covarine W3799” by the company | | |
| Wackherr | | |
| Diazolidinylurea | 0.39 | 0.39 |
| Perfluorohexyl betaine | 0.05 | 0.05 |
| Ethanol | 4.25 | 4.25 |
| Preserving agents | 0.54 | 0.54 |
| Demineralized water | qs 100 | qs 100 |

Evaluation of the Color Homogeneity

These varnishes were stored at 45° C. for two months:

The varnish of Example 4 showed good color homogeneity over time: after having been placed at 45° C. for 2 months, it remained pink from the top to the bottom of the bottle containing it.

The varnish of Example 5 showed colour heterogeneity after 2 months at 45° C.: it shows considerable white marbling along the wall of the bottle containing it.

### Examples 6 and 7

### Green-Shade Nail Varnishes

Two green-shade nail varnishes were prepared:
The varnish of Example 6 according to the present disclosure comprised the gelling polymer of Example 1.

The nail varnish of Example 7 (comparative) did not comprise a polymer comprising water-soluble units and units with an LCST, and comprised an associative polyurethane (Serad FX 1100).

| | Example 6 | Example 7 |
|---|---|---|
| Constituents | (Inventive) | Comparative |
| Polymer of Example 1 | 0.5 | |
| Associative polyurethane sold under the | | 0.08 |
| name “Serad FX 1100” by the company | | |
| Servo Huls | | |
| Aqueous polyurethane dispersion at a | 30.9 (AM) | 30.9 (AM) |
| solids content of 35% by weight, sold under | | |
| the name “Cydrothane HP 6000” by the | | |
| company Cytec | | |
| Blue organic dye sold under the name | 1.2 | 1.2 |
| “Bleu Covarine W 6795” by the company | | |
| Wackherr | | |
| Yellow organic dye sold under the name | 1.2 | 1.2 |
| “Jaune Covarine W 1796” by the company | | |
| Wackherr | | |
| Diazolidinylurea | 0.39 | 0.39 |
| Perfluorohexyl betaine | 0.05 | 0.05 |
| Ethanol | 4.25 | 4.25 |
| Preserving agents | 0.54 | 0.54 |
| Demineralized water | qs 100 | qs 100 |

Evaluation of the Color Homogeneity

The varnishes were stored at 45° C. for two months:

The varnish of Example 6 showed good color homogeneity over time: after having been stored at 45° C. for 2 months, it remained green from the top to the bottom of the bottle containing it.

The varnish of Example 7 showed color heterogeneity: it had a layer of yellow color at the bottom of the bottle containing it.

1. A nail varnish composition comprising, in a cosmetically acceptable aqueous medium, at least one polymer chosen from water-soluble and water-dispersible polymers, wherein the at least one polymer is such that the viscosity of the composition increases when it is subjected to a temperature above the gel point of the at least one polymer.

2. The nail varnish composition according to claim 1, wherein the at least polymer is such that, when the composition is subjected to a temperature of greater than or equal to the gel point of the at least one polymer +20° C., the viscosity of the composition increases by at least 10%, relative to its viscosity measured at the gel point of the polymer, for a shear rate equal to 10 s⁻¹.

3. The nail varnish composition according to claim 1, wherein the at least one polymer chosen from water-soluble and water-dispersible polymers comprises water-soluble or water-dispersible units, and units with a Lower Critical Solution Temperature (LCST), wherein the units with an LCST have a solution temperature in water ranging from 5° C. to 40° C., and are present in an amount by weight of 1%.

4. A nail varnish composition comprising, in a cosmetically acceptable aqueous medium, at least one polymer chosen from water-soluble and water-dispersible polymers, said at least one polymer comprising water-soluble or water-dispersible units and units with a Lower Critical Solution Temperature (LCST), wherein the units with an LCST have a solution temperature in water ranging from 5° C. to 40° C., and are present in an amount by weight of 1%.

5. The nail varnish composition according to claim 4, wherein the at least one polymer has a gel point ranging from 5° C. to 50° C., for an amount by weight in water of 2%.

6. The nail varnish composition according to claim 4, wherein the at least one polymer is in the form of a block polymer comprising water-soluble or water-dispersible units and alternating units with an LCST, or in the form of a grafted polymer comprising an optionally crosslinked skeleton formed from water-soluble or water-dispersible units and bearing grafts comprising units with an LCST, or in the form of a grafted polymer comprising an optionally crosslinked skeleton formed from LCST units and bearing grafts comprising water-soluble or water-dispersible units.

7. The nail varnish composition according to claim 4, wherein the water-soluble or water-dispersible units of the polymer are totally or partially formed by polymerization of at least one water-soluble monomer or a salt thereof chosen from:
(meth)acrylic acid;
vinylsulfonic acid;
(meth)allylsulfonic acid;
vinylphosphonic acid;
methylvinylimidazolium chloride;
(meth)acrylamide;
2-vinylpyridine or 4-vinylpyridine;
maleic acid and anhydride;
crotonic acid;
itaconic acid;
vinyl alcohols of formula CH₂═CHOH;
N-vinyllactams comprising a cyclic alkyl group of 4 to 9 carbon atoms;
water-soluble styrene derivatives;
dimethyldiallylammonium chloride;
N-vinylacetamide and N-methyl-N-vinylacetamide;
N-vinylformamide and N-methyl-N-vinylformamide;
vinyl monomers of formula (I): 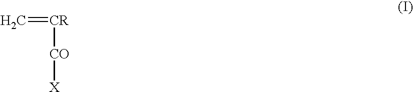
wherein:
R is chosen from H, —CH₃, —C₂H₅ and —C₃H₇ groups, and
X is chosen from:
alkyl oxides of —OR′ type wherein R′ is chosen from linear and branched, saturated and unsaturated hydrocarbon-based radicals comprising from 1 to 6 carbon atoms, substituted with at least one entity chosen from halogen atoms; sulfonic groups (—SO₃⁻), sulfate groups (—SO₄⁻), phosphate groups (—PO₄H₂⁻); hydroxyl groups (—OH); ether groups; primary amine groups (—NH₂); secondary amine groups (—NHR₁), tertiary amine groups (—NR₁R₂), and quaternary amine groups (—N⁺R₁R₂R₃) wherein R₁, R₂ and R₃, which may be identical or different, are chosen from linear and branched, saturated and unsaturated hydrocarbon-based radicals comprising from 1 to 6 carbon atoms, with the proviso that the sum of the carbon atoms of R′+R₁+R₂+R₃ does not exceed 7; and
—NH₂, —NHR₄ and —NR₄R₅ groups wherein R₄ and R₅, which may be identical or different, are chosen from linear and branched, saturated and unsaturated hydrocarbon-based radicals comprising from 1 to 6 carbon atoms, with the proviso that the total number of carbon atoms of R₄+R₅ does not exceed 7; wherein R₄ and R₅ are substituted with at least one entity chosen from halogen atoms; hydroxyl groups (—OH); sulfonic groups (—SO₃⁻); sulfate groups (—SO₄⁻); phosphate groups (—PO₄H₂⁻); primary amine groups (—NH₂); secondary amine groups (NHR₁), tertiary amine groups (—NR₁R₂) and/or quaternary amine groups (—N⁺R₁R₂R₃) wherein R₁, R₂ and R₃, which may be identical or different, are chosen from linear and branched, saturated and unsaturated hydrocarbon-based radicals comprising from 1 to 6 carbon atoms, with the proviso that the sum of the carbon atoms of R₄+R₅+R₁+R₂+R₃ does not exceed 7.

8. The nail varnish composition according to claim 4, wherein the water-soluble or water-dispersible units of the polymer are totally or partially formed by copolymerization of at least one hydrophobic monomer with at least one water-soluble monomer, wherein the hydrophobic monomer is present in an amount such that the resulting units are water-soluble or water-dispersible, the hydrophobic monomer being chosen from:
styrene and its derivatives;
vinyl acetate of formula CH₂═CH—OCOCH₃;
vinyl ethers of formula CH₂═CHOR₆, wherein R₆ is a linear or branched, saturated or unsaturated hydrocarbon-based radical comprising from 1 to 6 carbon atoms;
acrylonitrile;
caprolactone;
vinyl chloride and vinylidene chloride;
silicone derivatives;
hydrophobic vinyl monomers of formula (II): 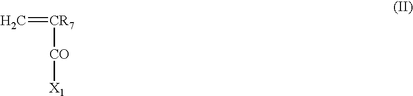
wherein:
R₇ is chosen from a hydrogen atom and —CH₃, —C₂H₅ and C₃H₇ groups,
X₁ is chosen from:
alkyl oxides of the type —OR₈ wherein R₈ is a linear or branched, saturated or unsaturated hydrocarbon-based radical comprising from 1 to 8 carbon atoms;
NH₂, —NHR₉ and —NR₉R₁₀ groups wherein R₉ and R₁₀, which may be identical or different, are chosen from linear and branched, saturated and unsaturated hydrocarbon-based radicals comprising from 1 to 6 carbon atoms, with the proviso that the total number of carbon atoms in R₉+R₁₀ does not exceed 6.

9. The nail varnish composition according to claim 7, wherein the water-soluble or water-dispersible units of the polymer are totally or partially neutralized with a mineral or organic base chosen from salts of sodium, ammonium, lithium, calcium, magnesium, ammonium substituted with 1 to 4 alkyl groups bearing from 1 to 15 carbon atoms, monoethanolamine, diethanolamine, triethanolamine, aminoethylpropanediol, N-methylglucamine, and basic amino acids.

10. The nail varnish composition according to claim 8, wherein the water-soluble or water-dispersible units of the polymer are totally or partially neutralized with a mineral or organic base chosen from salts of sodium, ammonium, lithium, calcium, magnesium, ammonium substituted with 1 to 4 alkyl groups bearing from 1 to 15 carbon atoms, monoethanolamine, diethanolamine, triethanolamine, aminoethylpropanediol, N-methylglucamine, and basic amino acids.

11. The nail varnish composition according to claim 4, wherein the water-soluble or water-dispersible units of the polymer are crosslinked by the action of at least one polyolefinically unsaturated compound chosen from divinylbenzene, diallyl ether, dipropylene glycol diallyl ether, polyglycol diallyl ethers, triethylene glycol divinyl ether, hydroquinone diallyl ether, tetrallyl-oxethanoyl, polyfunctional alcohol allyl and vinyl ether derivatives, tetraethylene glycol diacrylate, triallylamine, trimethylolpropane diallyl ether, tetraallyloxyethane, methylenebisacrylamide, allyl ethers of alcohols of the sugar series, allyl methacrylate and trimethylolpropane triacrylate (TMPTA).

12. The nail varnish composition according to claim 4, wherein the water-soluble or water-dispersible units of the polymer comprise at least one compound chosen from:
water-soluble polyurethanes,
xanthan gum,
alginates and derivatives thereof;
cellulose derivatives;
galactomannans and derivatives thereof; and
polyethyleneimine.

13. The nail varnish composition according to claim 4, wherein the water-soluble or water-dispersible units have a molar mass ranging from 1,000 g/mol to 10,000,000 g/mol when they constitute the water-soluble skeleton of a grafted polymer, or a molar mass ranging from 500 g/mol to 500,000 g/mol when they constitute a block of a multiblock polymer or when they constitute the grafts of a grafted polymer.

14. The nail varnish composition according to claim 4, wherein the units with an LCST comprise at least one polymer chosen from:
polyethers,
polyvinyl methyl ether,
polymeric and copolymeric N-substituted acrylamide derivatives with an LCST, and
poly-N-vinylcaprolactam and N-vinylcaprolactam copolymers.

15. The nail varnish composition according to claim 14, wherein the units with an LCST comprise random copolymers of ethylene oxide (EO) and of propylene oxide (PO) represented by the formula:
(EO)ₘ(PO)ₙ
wherein m is an integer ranging from 1 to 40, and n is an integer ranging from 10 to 60.

16. The nail varnish composition according to claim 15, wherein m ranges from 2 to 20, and n ranges from 20 to 50.

17. The nail varnish composition according to claim 15, wherein the units with an LCST have a molar mass ranging from 500 to 5,300 g/mol.

18. The nail varnish composition according to claim 17, wherein the units with an LCST have a molar mass ranging from 1,500 to 4,000 g/mol.

19. The nail varnish composition according to claim 14, wherein the units with an LCST are chosen from polymeric and copolymeric N-substituted acrylamide derivatives with an LCST.

20. The nail varnish composition according to claim 19, wherein the polymeric and copolymeric N-substituted acrylamide derivatives with an LCST are chosen from poly-N-isopropylacrylamide, poly-N-ethylacrylamide and copolymers of N-isopropylacrylamide or of N-ethylacrylamide and of a vinyl monomer chosen from those of formulae (I) or (II) below, or of a monomer chosen from (meth)acrylic acid, vinylsulfonic acid, (meth)allylsulfonic acid, maleic acid and anhydride, vinylphosphonic acid, crotonic acid, itaconic acid, (meth)acrylamide, vinylpyridine, vinyl alcohol, N-vinyllactams, styrene and its derivatives, dimethyldiallylammonium chloride, N-vinylacetamide, N-methyl-N-vinylacetamide, N-vinylformamide, N-methylvinylformamide, vinyl ethers, vinyl acetate, acrylonitrile, caprolactone, vinyl chloride and vinylidene chloride,
wherein the vinyl monomers of formula (I) have the formula: 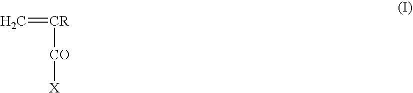
wherein:
R is chosen from H, —CH₃, —C₂H₅ and —C₃H₇ groups, and
X is chosen from:
alkyl oxides of —OR′ type wherein R′ is chosen from linear and branched, saturated and unsaturated hydrocarbon-based radicals comprising from 1 to 6 carbon atoms, substituted with at least one entity chosen from halogen atoms; sulfonic groups (—SO₃⁻), sulfate groups (—SO₄⁻), phosphate groups (—PO₄H₂⁻); hydroxyl groups (—OH); ether groups; primary amine groups (—NH₂); secondary amine groups (—NHR₁), tertiary amine groups (—NR₁R₂), and quaternary amine groups (—N⁺R₁R₂R₃) wherein R₁, R₂ and R₃, which may be identical or different, are chosen from linear and branched, saturated and unsaturated hydrocarbon-based radicals comprising from 1 to 6 carbon atoms, with the proviso that the sum of the carbon atoms of R′+R₁+R₂+R₃ does not exceed 7; and
—NH₂, —NHR₄ and —NR₄R₅ groups wherein R₄ and R₅, which may be identical or different, are chosen from linear and branched, saturated and unsaturated hydrocarbon-based radicals comprising from 1 to 6 carbon atoms, with the proviso that the total number of carbon atoms of R₄+R₅ does not exceed 7; wherein R₄ and R₅ are substituted with at least one entity chosen from halogen atoms; hydroxyl groups (—OH); sulfonic groups (—SO₃⁻); sulfate groups (—SO₄⁻); phosphate groups (—PO₄H₂⁻); primary amine groups (—NH₂); secondary amine groups (NHR₁), tertiary amine groups (—NR₁R₂) and/or quaternary amine groups (—N⁺R₁R₂R₃) wherein R₁, R₂ and R₃, which may be identical or different, are chosen from linear and branched, saturated and unsaturated hydrocarbon-based radicals comprising from 1 to 6 carbon atoms, with the proviso that the sum of the carbon atoms of R₄+R₅+R₁+R₂+R₃ does not exceed 7; and
the vinyl monomers of formula (II) have the formula: 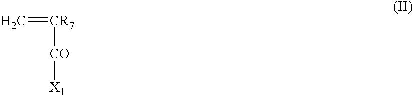
wherein:
R₇ is chosen from a hydrogen atom and —CH₃, —C₂H₅ and C₃H₇ groups,
X₁ is chosen from:
alkyl oxides of the type —OR₈ wherein R₈ is chosen from linear and branched, saturated and unsaturated hydrocarbon-based radicals comprising from 1 to 8 carbon atoms;
NH₂, —NHR₉ and —NR₉R₁₀ groups wherein R₉ and R₁₀, which may be identical or different, are chosen from linear and branched, saturated and unsaturated hydrocarbon-based radicals comprising from 1 to 6 carbon atoms, with the proviso that the total number of carbon atoms in R₉+R₁₀ does not exceed 6.

21. The nail varnish composition according to claim 14, wherein the derivatives have a molar mass ranging from 1,000 g/mol to 500,000 g/mol.

22. The nail varnish composition according to claim 19, wherein the units with an LCST comprise a poly-N-vinylcaprolactam, or a copolymer of N-vinylcaprolactam and of a vinyl monomer chosen from those of formulae (I) and (II), or of a monomer chosen from (meth)acrylic acid, vinylsulfonic acid, (meth)allylsulfonic acid, maleic acid, maleic anhydride, vinylphosphonic acid, crotonic acid, itaconic acid, (meth)acrylamide, vinylpyridine, vinyl alcohol, N-vinyllactams, styrene and its derivatives, dimethyldiallylammonium chloride, N-vinylacetamide, N-methyl-N-vinylacetamide, N-vinylformamide, N-methylvinylformamide, vinyl acetate, vinyl ethers, acrylonitrile, caprolactone, vinyl chloride and vinylidene chloride.

23. The nail varnish composition according to claim 22, wherein the units with an LCST have a molar mass ranging from 1,000 g/mol to 500,000 g/mol.

24. The nail varnish composition according to claim 3, wherein the units with an LCST are present in an amount ranging from 5% to 70% by weight, relative to the weight of the polymer.

25. The nail varnish composition according to claim 24, wherein the units with an LCST are present in an amount ranging from 30% to 60% by weight, relative to the total weight of the polymer.

26. The nail-varnish composition according to claim 4, wherein the units with an LCST are present in an amount ranging from 5% to 70% by weight, relative to the total weight of the polymer.

27. The nail varnish composition according to claim 26, wherein the units with an LCST are present in an amount ranging from 30% to 60% by weight, relative to the total weight of the polymer.

28. The nail varnish composition according to claim 3, wherein the solution temperature of the units with an LCST ranges from 10° C. to 35° C., when the units with an LCST are present in water in an amount of 1% by weight.

29. The nail varnish composition according to claim 4, wherein the solution temperature of the units with an LCST ranges from 10° C. to 35° C., when the units with an LCST are present in water in an amount of 1% by weight.

30. The nail varnish composition according to claim 3, wherein the at least one polymer comprising units with an LCST is present in a solids amount ranging from 0.01% to 20% by weight, relative to the total weight of the composition.

31. The nail varnish composition according to claim 30, wherein the at least one polymer comprising units with an LCST is present in a solids amount ranging from 0.1% to 15% by weight, relative to the total weight of the composition.

32. The nail varnish composition according to claim 4, wherein the at least one polymer comprising units with an LCST is present in a solids amount ranging from 0.01% to 20% by weight, relative to the total weight of the composition.

33. The nail varnish composition according to claim 32, wherein the at least one polymer comprising units with an LCST is present in a solids amount ranging from 0.1% to 15% by weight, relative to the total weight of the composition.

34. The nail varnish composition according to claim 3, further comprising at least one additional film-forming polymer.

35. The nail varnish composition according to claim 4, further comprising at least one additional film-forming polymer.

36. The nail varnish composition according to claim 34, wherein the at least one additional film-forming polymer is present in a solids amount ranging from 5% to 70% by weight, relative to the total weight of the composition.

37. The nail varnish composition according to claim 36, wherein the at least one additional film-forming polymer is present in a solids amount ranging from 20% to 50% by weight, relative to the total weight of the composition.

38. The nail varnish composition according to claim 35, wherein the at least one additional film-forming polymer is present in a solids amount ranging from 5% to 70% by weight, relative to the total weight of the composition.

39. The nail varnish composition according to claim 38, wherein the at least one additional film-forming polymer is present in a solids amount ranging from 20% to 50% by weight, relative to the total weight of the composition.

40. A nail varnish composition according to claim 1, wherein the aqueous medium is present in an amount ranging from 5% to 95% by weight, relative to the total weight of the composition.

41. A nail varnish composition according to claim 40, wherein the aqueous medium is present in an amount ranging from 20% to 80% by weight, relative to the total weight of the composition.

42. A nail varnish composition according to claim 4, wherein the aqueous medium is present in an amount ranging from 5% to 95% by weight, relative to the total weight of the composition.

43. A nail varnish composition according to claim 42, wherein the aqueous medium is present in an amount ranging from 20% to 80% by weight, relative to the total weight of the composition.

44. The nail varnish composition according to claim 1, further comprising at least one dyestuff.

45. The nail varnish composition according to claim 4, further comprising at least one dyestuff.

46. The nail varnish composition according to claim 44, wherein the at least one dyestuff is present in an amount ranging from 0.01% to 10% by weight, relative to the total weight of the composition.

47. A non-therapeutic cosmetic process for making up and/or caring for the nails, comprising the application to the nails of at least one coat of a nail varnish composition comprising, in a cosmetically acceptable aqueous medium, at least one polymer chosen from water-soluble and water-dispersible polymers, wherein the at least one polymer is such that the viscosity of the composition increases when it is subjected to a temperature above the gel point of the at least one polymer.

48. A non-therapeutic cosmetic process for making up and/or caring for the nails, comprising the application to the nails of at least one coat of a nail varnish composition comprising, in a cosmetically acceptable aqueous medium, at least one polymer chosen from water-soluble and water-dispersible polymers, wherein the least one polymer comprises water-soluble or water-dispersible units and units with a Lower Critical Solution Temperature (LCST), wherein the units with an LCST have a solution temperature in water ranging from 5° C. to 40° C., and are present in an amount of 1% by weight.

49. A method for imparting color homogeneity over time to nail varnish compositions, comprising adding to a nail varnish composition comprising a cosmetically acceptable aqueous medium, at least one polymer chosen from water-soluble and water-dispersible polymers, wherein said at least one polymer comprises water-soluble or water-dispersible units, and units with a Lower Critical Solution Temperature (LCST), wherein the units with an LCST have a solution temperature in water ranging from 5° C. to 40° C., and are present in an amount of 1% by weight.
